(19) United States
(12) Patent Application Publication
(10) Pub. No.: US 2020/0238626 A1
(43) Pub. Date: Jul. 30, 2020

(54) Custom Orthotics and Personalized Footwear

(71) Applicant: NIKE, Inc., Beaverton, OR (US)

(72) Inventors: David Bleicher, Tel-Aviv (IL); Tamir Lousky, Bat-Yam (IL)

(21) Appl. No.: 16/632,601

(22) PCT Filed: Jul. 21, 2017
(86) PCT No.: PCT/US17/43373

§ 371 Date: Jan. 21, 2020

(51) Int. Cl. B29C 64/393; A61F 5/14; G06F 30/12; B33Y 50/02; B33Y 10/00; B33Y 30/00; A43B 17/14

(52) U.S. Cl. CPC B29C 64/393; A61F 5/14; G06F 30/12; G06F 2113/22; B33Y 10/00; B33Y 30/00; A43B 17/14; B33Y 50/02

(57) ABSTRACT

A system, process, manufacturing technique and platform are herein provided for designing and/or manufacturing orthopedic devices, custom orthotics or personalized footwear based on computerized design software adapted to adjust the scanned information into a 3D model of the device substantially ready to production, wherein the system comprises an imaging module which uses image recognition to identify different anatomic parts of the foot to allow the design of the orthotics, and a human interface that allows showing the original scan of the foot at an opaque or semitransparent manner to allow visualization of the way the foot is going to fit and be supported by the designed custom orthotic.

### FIELD OF THE INVENTION

The present invention relates to the field of orthotics, more specifically to systems and processes for custom orthotics.

### BACKGROUND

Foot orthotics are designed to treat foot or arch pain by providing cushioning, stability or support, sometimes attempting to adjust or stabilize foot movement. Prior to about 1950, there was no standardization in the methods used to treat foot pain. A standardized approach to the design of foot orthotics was introduced in 1954, when Merton L. Root, DPM, revolutionized the field with the theory of the Subtalar Neutral Position (STNP).

The subtalar joint is the joint between the talus and calcaneus bones. Subtalar neutral is where the subtalar joint is neither pronated nor supinated and its importance was based on observations of what Root subjectively deemed to be “normal” feet. According to Root's theory, correction of a foot to a “normal” position involves placing of only the subtalar joint into a ‘neutral’ position, the so-called subtalar neutral position or STNP.

There are two basic types of custom orthotics: accommodative orthotics which are built to cushion the foot and remove pain and functional orthotics which are used to treat the patient by repositioning the foot at a certain position. An accommodative orthotic is typically made from a soft or flexible material that “accommodates” any deformity of the foot. This cushioning orthotic also results in some dissipation of the forces required for efficient gait that ordinarily would be transmitted up the kinetic chain. A functional orthotic is one that is built to control foot position and joint movement. These orthotics are typically built from rigid materials, and are used by clinicians to hold the foot in a position they deem therapeutic. This may be problematic because it does not allow the foot to continually adapt to the ground and operate efficiently.

For the manufacture of both types of orthoses, the plantar surface of the patient's foot is captured and its mirror image is produced on the surface of the orthotic device that contacts the patient's foot. Functional orthotics, abnormally maintain the foot's arch throughout gait, with the orthotic supporting the weight of the whole body and compressing the soft tissue between the bones and the orthotics rigid surface.

26 million people or in the United States that suffer from diabetes consist of 8% of the whole population. Diabetes complications include nerve damage and poor blood circulation in peripheral organs. These problems make the feet specifically vulnerable to skin sores (ulcers) and wounds that can get infected worsen quickly and are difficult to treat.

Diabetic foot complications are the most common cause of non-traumatic lower extremity amputations. The majority of diabetic foot complications begin with the formation of skin ulcers on the bottom of the foot.

One of the main causes for diabetic ulceration is the increase in plantar pressure on specific areas located on the bottom surface of the foot. Foot deformities, which are common in diabetic patients, lead to focal area high pressure zones. When coupled with lack of sensation, a foot ulcer can develop. Proper leveling of the weight over the lower surface of the foot as well as off-loading plantar foot pressure at ulceration points and wounds is an important component in treating diabetic foot and a factor of great importance in the preservation of these patient feet health. It is common for diabetic foot patient to be treated with cushioning accommodative orthotics. Although these are affective to some extension, they do not supply a solid structure to rebalance the patient's posture and thus do not rearrange the load leveling on the patient's foot, and fail to relieve the pressure from the focused pressure points of a patient's plantar surface. For patients who already suffer from ulceration and foot wounds to some extent, there exist insoles which consist of an array of retractable parts, which are adjusted to accommodate a patient's wounds when removed in the correct position. This solution provides a limited level of control for such patients in terms of the exact position to remove parts as well as the stiffness level and 3D geometry around these. Additionally these do not supply a correction to the foot posture and gait that will allow correct weight distribution and prevent from new wounds to occur.

The use of custom made orthotics which are inserted into the bottom of patients shoes is known in the art for various applications. Some of which are designed for helping certain foot, ankle, knee or back disorders or pain, others for constraining a certain foot posture and gait modifications.

Historically, patients who use prescription orthotic devices in their shoes are forced to temporarily give up the corrective or stabilizing orthotics if they want to wear sandals, slippers, clogs or flip-flops.

The use of orthotics with open shoes such as flip-flops or sandals is impossible due to the nature of the open form shape of these apparels. Nevertheless, many of the patients which depend on these orthotics, end up compromising on the orthopedic support when using sandals in a wet environment, or in hot weather in the summer.

Manufacturing technologies which are used for making sandals, flip-flops and sandals have improved over the years and allow great material, shape and colors. Although all of these depend on mass production tooling and technologies and can therefore not fit specifically to each patient's needs and specific foot shape. To date, there does not exist a manufacturing process which allows personalized manufacturing of sandals, clogs and flip-flops that are specifically built according to each of the patient's needs while retaining the advantages such as an open elegant shoe, comfortable in the summer and which can be wet without absorbing water.

Because there are no two feet that are alike, it is reasonable for every person to use footwear which is personally designed to his own feet. As for today, there exist various footwear manufactured according to specified length sizes and sometimes also width sizes. Nevertheless there does not exists footwear which are specifically formed to a consumers size and form shape of a foot and which ensures the correct posture of ones feet during weight baring and gait without the need of an additional insert type orthotic.

### SUMMARY

The current invention relates to personalized orthotic and customized footwear aimed to address patients suffering from diabetic foot as well as other clinical conditions related with the body's lower extremity as well as systems and processes for generation and manufacture thereof. More specifically, embodiments of the invention herein describes novel manufacturing systems and methods for custom orthotics and personalized footwear which allow the design of the soles to selectively redistribute the loads and stress points on a patients foot and place strain relieving areas underneath ulcers and wounds in order to inhibit blood circulation and facilitating curing of these. An embodiment of a system and process includes a 3D mapping unit which captures morphological as well as spectral data from the patient's foot and transmits the resulting image data via the internet. In exemplary process, the image data capture is performed while the foot is held in the corrected neutral position and retained in this posture using a positioning device. The system automatic amends the received image data or receives manual amendment input to the foot shape according to the transmitted information in order to adjust the foot to the correct position which allows best weight distribution and correct posture of the foot. The negative form of the received and optionally amended shape is transferred into an automatic routing machine or 3D additive manufacturing machine which creates one or more mold inserts with the exact shape of the patient's orthotics derived from the image capture data. The molds are injected using a foamed material of a suitable composition for a desired weight, density, hardness, color, and other material properties. The injected orthotics may be used as personalized insoles in existing shoes, or be integrated in personalized sandals, clogs, flip-flops, and additional applications.

These and other features, aspects, and advantages of the invention will become better understood with reference to the following description, appended claims, and accompanying drawings.

A system is provided herein for designing orthopedic devices, custom orthotics or personalized footwear based on computerized design software adapted to adjust the scanned information into a 3D model of the device substantially ready to production, the system comprising an imaging module which uses image recognition to identify different anatomic parts of the foot to allow the design of the orthotics.

The system as described may use the spectral information from the scanned data in order to manually or automatically recognize any wound, ulceration or other defects on the surface or internal part of the foot and uses this information to redistribute the loads on the foot and release stress from these portions.

In some embodiments, the spectral information is filtered and the IR and NIR spectral range which used to determine “hot spots” and strain points which are not obvious from the regular spectral analysis of the plantar surface of the foot.

In some embodiments, the system recognizes markers on the foot to identify the portions of the foot as well as but not limited to corrected posture, angles and positions.

In some embodiments, the system uses the data from the markers to log the position of specific anatomical markers or portions and uses this information to calculate the correct position of the foot on top of the orthotic or inside the personalized shoe/sandal.

In some embodiments, the system uses anatomical markers automatically recognized along the foot to track the location instead of the added markers.

In some embodiments, the system allows manual sculpting operations on specific areas on the model such as: inflation/deflation, smoothing, gradual pulling/pushing operations based on the strength and area of operation described by the user.

In some embodiments, the system integrates data inserted by the physician or care giver regarding the age, height, weight, clinical condition, sports engagements, occupation, type of shoes worn or any additional relevant information to automatically make decision regarding the design and form of the orthotic device being built.

In some embodiments, the system limits the technician ranges of operations according to the information collected from the caregiver.

In some embodiments, the system utilizes in addition to the scanned feet of a patient a scan of his intended shoes or the existing insoles he is using. This information is used by the system to determine the cutting shape and size for the orthotics in order to fit exactly into the shoes of the specified patient.

In some embodiments, the system includes saved shapes of cutting according to existing insoles, shoe sizes and specifically to fit relevant application described above such as personalized clog inner parts, one piece inner clogs, flip flops, sandals or personalized shoes.

In some embodiments, the system includes a human interface that allows showing the original scan of the foot at an opaque or semitransparent manner to allow visualization of the way the foot is going to fit and be supported by the designed custom orthotic.

In some embodiments, the system includes a human interface which includes section view of the plantar surface, the designed insole, the original scan or any combination of the above to evaluate the design and fit at different sections of the insoles/shoe.

In some embodiments, the system includes functionality that allows translation of the positive volume of the designed insole into a carved negative model “mold” intended for injection of the material to form the device.

In some embodiments, the system adds a revolved lip surface around the cavity of the formed mold in order to allow the mold to close and seal against a flat surface in the process of injection molding.

In some embodiments, the system allows a geometrical link of two or more mold blocks in order to facilitate mass production of these using a 3D automated milling machine, 3D printer or alternative 3D manufacturing automated technologies.

In some embodiments, the system automatically or manually builds an array or matrix of molds on two sides of a block or plurality of blocks to allow mass production.

In some embodiments, the system tests the protrusion levels of molds on two sides of a block to ensure they do not protrude into the volume of each-other.

In some embodiments, the system adds information relevant to the patient, orthotic or mold directly onto the model and then to the CNC. This information may comprise of the name of the patient, the date, the volume or other measures of the mold which may or may not be relevant to the injection molding process.

In some embodiments, the system includes functionality that allows control over the surface of the device and enables a flat smooth device as well as additional geometrical shapes such as stripes, protruding spheres, pyramid shapes or any other shape.

In some embodiments, the system automatically distributes the surface shapes onto the surface of the device according to an even spread or a specified spread. This spread is automatically adjusted to fit the shape of the specific orthotic.

In some embodiments, the system allows positioning of specific additional geometrical protrusions onto the surface of the orthotic such as a “bridge” located under the fingers to support and allow a better grip or a protruding shape or volume which are meant to change the weight distribution and reduce stress from specified areas.

In some embodiments, the system allows positioning of specific Holes or depressed areas on the surface of the orthotic. The can be used to reduce pressure from specific areas as well as to avoid contact of specific section of the foot, such as wounds or ulcers with the bottom of the insole.

A marker system is herein provided for marking segments on the skin of a patient which are recognized using the system and are used for the identification of the specific organ parts as well as required posture, the marker system comprising stickers which are easily recognized using the 3D scanner according to color information, specified geometry, and/or light absorption properties.

In some embodiments, the marker system includes markers that are painted onto the skin of a patient which are easily recognized using the 3D scanner according to color information, specified geometry, light absorption properties or any combination of the above.

In some embodiments, the system enables designing 3D models or objects which are specifically adjusted to fit a certain portion of the human body, referring to other clinical applications than foot/orthotics. The system may include to all the features and details which are relevant to the application.

In some embodiments, the system is specifically used as head fixation device to support the head of patients.

In some embodiments, the system is used as support devices for fractured bones on limbs.

In some embodiments, the system is built to support a fractured or injured leg of a patient.

In some embodiments, the system is built to support a fractured or injured hand of a patient.

In some embodiments, the system is used for holding a patient in a specific position in an operation room, hospital, bed or chair.

In some embodiments, the system is used for orthopedic rigid, semi rigid or fully moving braces for the foot, ankle, knee, back, neck or elbow made based on these capabilities.

In some embodiments, the system is used for holding designing personalized apparatus that comes in direct contact with the body such as a chair or mattress.

In some embodiments, the system is used as support devices for fractured bones on limbs.

A manufacturing technique is herein provided for building custom orthotic insoles or personalized sandals or footwear soles using injection of a foamed or soft material into a mold.

In some embodiments, the manufacturing process includes a mold that consists of a “mold house” which is a tool that includes heating circles, cooling tubes if necessary and a fixed flat side as well as a cavity built to receive inserts according to the geometry of a specific orthotic.

In some embodiments, the manufacturing process is described where each insert has one leg of a specific patient on each side of the block, allowing a two sided block to contain all of a patient's devices.

In some embodiments, the manufacturing process is described where the block is injected at a vertical orientation allowing flow of the materials and filling of the whole cavity.

In some embodiments, the manufacturing process is described where the blocks contain a “air pocket” at the rear of the orthotic which is at the upper most side of the block during vertical injection. The pocket is connected to the orthotics' cavity allowing all the air bubbles to release from the orthotic and achieving a bubbles free surface on the device.

In some embodiments, the manufacturing process as described in claim 4.1 where the blocks contain a “air pocket” at the rear of the orthotic which is at the upper most side of the block during vertical injection. The pocket is connected to the orthotics' cavity allowing all the air bubbles to release from the orthotic and achieving a bubbles free surface on the device.

In some embodiments of the manufacturing process, the blocks include information which is manually or automatically inserted into the injection system such as volume, hardness level, injection time as well as personal information such as but not limited to patient name, serial number or bar code information.

In some embodiments of the manufacturing process, the blocks are made using additive manufacturing technologies such as 3D printing according to a model designed using the system as described in claim 1.

In some embodiments of the manufacturing process, the materials injected are foamed polyurethane, polyethylene, EVA, foamed PVC or silicone.

In some embodiments of the manufacturing process, the materials injected are single component materials.

In some embodiments of the manufacturing process, the materials injected are made from a composition of two or more components having a chemical reaction curing the material and causing the foaming inside the mold.

In some embodiments of the manufacturing process, the ratio between the different components of the material can be controlled allowing control over the density, weight, hardness or any combination of the above of the injected product.

In some embodiments of the manufacturing process, inserted elements are placed inside the mold cavity prior to injection and form shapes on volumes which are not controlled in the mold fabrication.

In some embodiments of the manufacturing process, inserted elements are placed inside the mold and become part of the molded product once the mold is injected with material.

A manufacturing of composite material orthotics is herein described, in which the inserts are made from, foamed polyurethane, polyethylene, EVA, foamed PVC or silicone or any combination of the above.

In some embodiments, the manufacturing process includes two or more injection steps of similar or different materials, creating a multilayer composite material of varying properties which are adhered together in a single component device.

Custom orthotic insoles are herein described, that are designed to treat foot wounds and ulcerations by correct distribution of the weight of a patient along the plantar surface of the foot and by including strain release depressed areas/holes in the orthotic.

In some embodiments, the custom orthotic insoles include a plurality of materials of different hardness levels. The soft materials are located under the wounds allowing load reduction from these points.

In some embodiments, the custom orthotic insoles enable treatment for diabetic foot ulceration, or foot wounds or wound infection or any combination of the above using a series of orthotic insoles. As the wounds begin to heal, the depression and holes which are located under the center areas of the ulcerous wounds are filled by torus shaped inserts which reduce the diameter of these. These supporting additions are added in two to four steps until the wound is fully healed.

In some embodiments, the custom orthotic insoles enable treatment for diabetic foot ulceration, or foot wounds or wound infection or any combination of the above using a series of orthotic insoles. The orthotic pairs are differed from each other by the size of the depressions. As the wounds begin to heal, the patient will change between the orthotics, until wounds are healed and no depression are needed.

In some embodiments, the custom orthotic insoles include depression areas that are coated with drugs which accelerate healing of the wounds.

In some embodiments, the custom orthotic insoles drug layer is encapsulated for slow release over a period of one week to six months.

In some embodiments, the custom orthotic insoles drug layer is coated fully or selectively with a anti-bacterial or anti-fungal layer.

In some embodiments, the custom orthotic insoles drug layer includes coating that is located at the depression areas located below the wounds and ulcer portions of a patient's foot.

In some embodiments, the custom orthotic insoles drug layer is coated fully or selectively with a silver particle based anti-bacterial coating.

In some embodiments, the custom orthotic insoles drug layer is supplied in multiple copies to allow patients to replace often in order to control infection factors, and improve healing process of the wounds.

In some embodiments, the custom orthotic insoles drug layer contains liquid drainage through the depression holes to a reservoir which is located at a distal surface from the wounds, thus allowing wounds to remain free of exudates and improving healing.

A custom orthotic flip-flop is herein provided, which is built according to specific mapping of a person's foot geometry in order to fit the plantar surface and supply arch support.

In some embodiments, the custom flip-flop includes scanning the patient's foot at a subtalar joint neutral position to correct the posture on a patient's foot.

In some embodiments, the custom flip-flop includes contains surface geometry such as spherical protrusions to allow massaging of a patient's foot as well as reducing the sweating and allowing airing of the lower surface of a patient.

In some embodiments, the custom flip-flop includes geometry that is spread according to a specific configuration, locating the protrusion areas at specific positions, according to a reflexology map of a patient or any other consideration.

In some embodiments, the custom flip-flop is built using a single molded part, specifically designed according to a patient's geometrical form.

In some embodiments, the custom flip-flop includes is built from one or more parts which include a cavity, designed to receive an orthotic insert, specific to a patient's design.

In some embodiments, the custom flip-flop includes includes one or more fixation elements at the heel part of the foot to improve the way the flip-flop connects to the foot.

In some embodiments, the custom flip-flop includes a fixation element that is detachable from the flip-slop and can be reattached.

Custom orthotic clogs are herein provided, which are built according to specific mapping of a person's foot geometry in order to fit the plantar surface and supply arch support.

In some embodiments, the custom orthotic clogs include data from the patient's foot scan that was performed at a subtalar joint neutral position to correct the posture on a patient's foot.

In some embodiments, the custom orthotic clogs include surface geometry such as spherical protrusions to allow massaging of a patient's foot as well as reducing the sweating and allowing airing of the lower surface of a patient.

In some embodiments, each custom orthotic clog is built using a single molded part, specifically designed according to a patient's geometrical form.

A mold is herein provided, for making the custom orthotic clogs, that includes a fixed cavity while part or the whole of the mold core is interchangeable according to inserts built to fit each patient's feet.

In some embodiments, the custom orthotic clogs are built from one or more parts which include a cavity, designed to receive an orthotic insert, specific to a patient's design.

In some embodiments, the custom orthotic clogs include inserts that are adhered or chemically bonded or heat molded to place in a water sealed manner to create a single part clog. The inserts will be made from the same color and material as the clog body or from different materials and colors.

In some embodiments, the custom orthotic clogs include inserts that are not physically connected in place. The insert can be replaced periodically according to different hardness levels, colors etc. The inserts will be made from the same color and material as the clog body or from different materials and colors.

Custom orthotic sandals are herein provided, which are built according to specific mapping of a person's foot geometry in order to fit the plantar surface and supply arch support.

In some embodiments, the custom orthotic sandals incorporate a patient's foot scan that was performed at a subtalar joint neutral position to correct the posture on a patient's foot.

In some embodiments, the custom orthotic sandals contain surface geometry such as spherical protrusions to allow massaging of a patient's foot as well as reducing the sweating and allowing airing of the lower surface of a patient.

In some embodiments, each of the custom orthotic sandals includes one or more parts which include a cavity, designed to receive an orthotic insert, specific to a patient's design. With a known external contour surface.

In some embodiments, the custom orthotic sandals include inserts that are adhered or chemically bonded or heat molded to place in water tight manner. The inserts will be made from the same color and material as the clog body or from different materials and colors.

In some embodiments, the custom orthotic sandals include inserts that are not physically connected in place. The insert can be replaced periodically according to different hardness levels, colors etc. The inserts will be made from the same color and material as the body or from different materials and colors.

Custom sports shoes are herein provided, including casual footwear, or special application shoes such as climbing shoes, ski boots, which are designed and manufacturing specifically according to a customer's foot geometry as well as additional parameters such as height, weight clinical condition or any other relevant factor.

In some embodiments, the custom footwear includes an inner part of the sole that is manufactured specifically to a patient's need using the manufacturing process described in claim 4.

In some embodiments, the custom footwear includes a sole that is made from a single part molding process, using an insert mold specific to a patient's geometry.

In some embodiments, the custom footwear includes a sole that is made from a multi phased over-molding process, using an insert mold specific to a patient's geometry. And combining it with the sole molds to allow multiple colors, material properties and geometrical factors.

In some embodiments, the custom footwear includes a sole that is made from multiple parts, some of which are made specifically according to a patient's needs and assembled together using chemical bonding, heat bonding or adhesive materials.

A process is herein provided for creating customized orthotics, wherein the process includes: receiving a 3D file of a foot, said 3D file comprising the metatarsal region, the arch region, and the heel region; detecting and assigning position data in the 3D file for the metatarsal region, the arch region, and the heel region; generating a base orthotic model, where the orthotic base model represents a surface for mating to the corresponding mapped plantar surface, the base orthotic model conforming to the mapped plantar surface.

In some embodiments, the process further comprises providing a body position device, operable to facilitate a constant foot position during 3D image data capture.

In some embodiments, the process further comprises providing a marker pen and marking the metatarsal region, the arch region, and the heel region.

In some embodiments, the process further comprises providing paint and marking the metatarsal region, the arch region, and the heel region.

In some embodiments, the process further comprises labeling and marking the metatarsal region, the arch region, and the heel region.

In some embodiments, the process further comprises providing a 3D scanner comprising a depth and color camera.

In some embodiments, the process further comprises using a Kinect camera.

In some embodiments, the process further comprises using a Primesense camera.

In some embodiments, the process further comprises using a Davis Laser camera.

In some embodiments, the process further comprises detecting and assigning position data in the 3D file for wounds on the foot.

In some embodiments, the process further comprises detecting using hotspot detection.

In some embodiments, the process further comprises enabling detection by color differentiation.

In some embodiments, the process further comprises modifying the base orthotic position to provide recesses corresponding to the position of the wounds.

In some embodiments, the process further comprises applying a healing agent as a substrate in the recesses.

In some embodiments, the process further comprises providing an exit channel in fluid communication with the recess and a remote reservoir.

In some embodiments, the process further comprises a plurality of concentrically disposed, successively smaller plugs dimensioned for insertion into the recess.

In some embodiments, the process further comprises wherein base orthotic model comprises a first section of a first angular orientation continuously joined to a transition section of a second orientation joined to a third section of a third orientation.

In some embodiments, the process further comprises presenting an interface for manipulation or verification of the base orthotic model.

In some embodiments, the process further comprises presenting an interface for one of the following: an inflation or deflation operation, a smoothing operation, a stretch or compress operation, and a rotation operation.

In some embodiments, the process further comprises generating the negative impression of the orthotic model and transforming into a model for a mold.

### BRIEF DESCRIPTION OF THE DRAWINGS

FIG. 1 illustrates a flowchart of embodiments of processes according to the current invention;

FIG. 2 illustrates a pictorial chart of embodiments of a process according to the current invention;

FIGS. 3a and 3b illustrate feet having wounds and anatomical markers;

FIG. 4 illustrates an embodiment of an orthotic of the current invention;

FIGS. 5a and 5b illustrates example orthotic amendments of a subprocess of the current invention;

FIGS. 6a and 6b illustrates alternate example orthotic amendments of a subprocess of the current invention;

FIG. 7a illustrates another example foot model amendment of a subprocess of the current invention;

FIG. 7b illustrates an example subset of a repository of available insoles;

FIGS. 8a and 8b illustrate an embodiment of an orthotic of the current invention in an alternate state;

FIG. 9 illustrates a foot having wounds;

FIG. 10 illustrates an embodiment of an orthotic of the current invention having a recess;

FIG. 11 illustrates an embodiment of an orthotic of the current invention having a recess;

FIG. 12a illustrates an embodiment of an orthotic of the current invention having a recess;

FIG. 12b illustrates a plurality of inserts for the orthotic embodiment of FIG. 12a;

FIGS. 13a and 13b illustrate alternate configurations of the orthotic embodiment of FIG. 11;

FIG. 14 illustrates an embodiment of a mold of the current invention;

FIG. 15a illustrates an alternate embodiment of a mold of the current invention;

FIG. 15b illustrates another alternate embodiment of a mold of the current invention;

FIGS. 16a-c illustrate embodiments of open footwear having orthotics of the current invention;

FIGS. 17a-c illustrate alternate embodiments of open footwear having orthotics of the current invention;

FIG. 18 illustrates an example 3D scanner subsystem of the current invention;

FIG. 19 illustrates an example 3D scanner subsystem of the current invention as it may exist in operation;

FIG. 20 illustrates a combined block and flow diagram of a molding process of the current invention;

FIG. 21 illustrates the mold of FIG. 15a as it may exist in operation; and

FIG. 22 illustrates an overview of a processes of the current invention.

### DETAILED DESCRIPTION

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. The description taken with the drawings making apparent to those skilled in the art how the several forms of the invention may be embodied in practice.

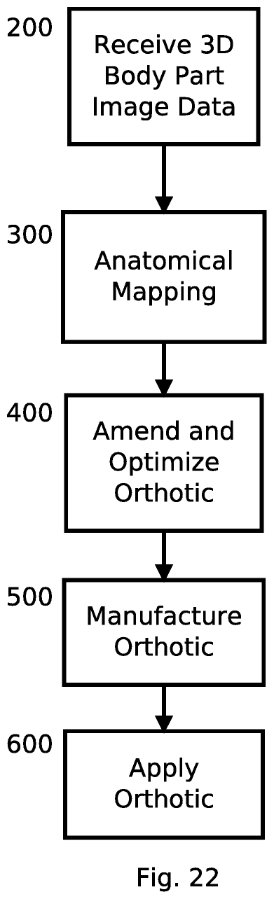

FIG. 22 illustrates an overview of certain embodiments of the invention. At step 200, the system receives 3D image data of a body part. At step 300, the system processes the received 3D image body for anatomical mapping. At step 400, the anatomically mapped image data is amended and optimized. At step 500, orthotics are manufactured based on the amended and optimized anatomically mapped image data. At step 600, the personalized manufactured orthotics are applied to footwear. More consideration will be given to each of these steps below.

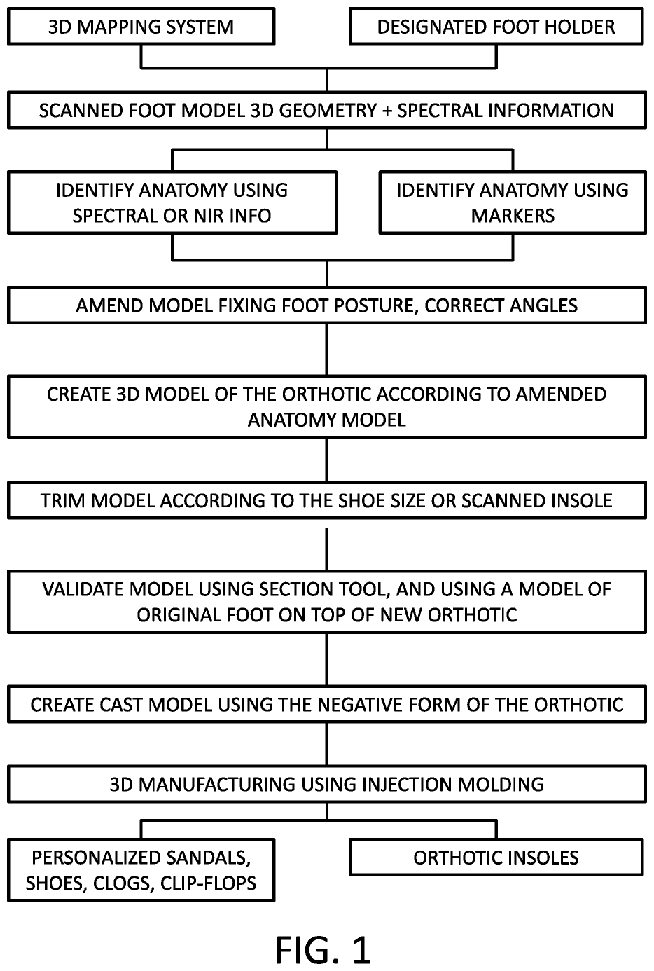

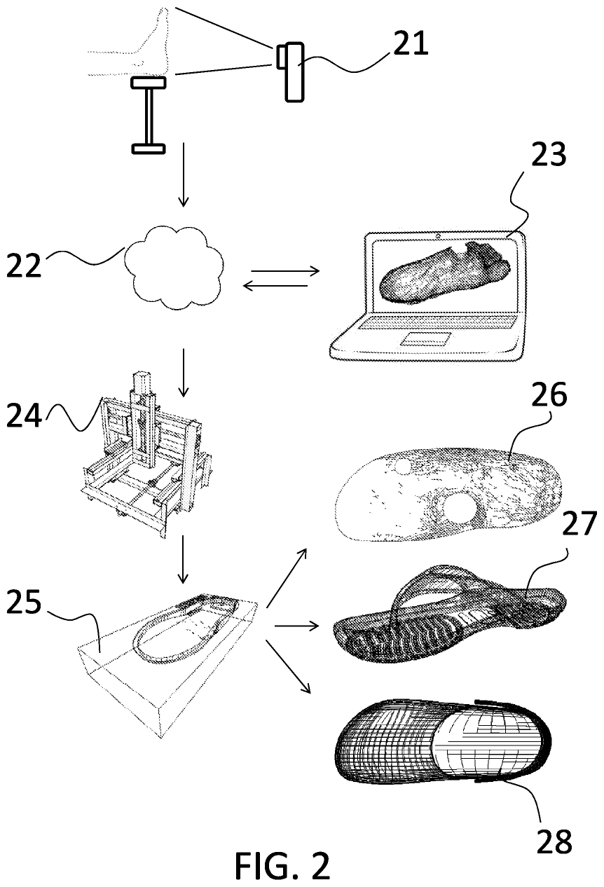

FIG. 1 shows a flow chart of embodiments of systems and processes of the current invention, while FIG. 2 depicts a pictoral view of a system and process of the current invention.

At step 200, the system receives 3D image data for a body part. The selected body part for image data capture in this description is the foot, although it is within the scope of this invention to apply the systems and processes of the invention to other body parts. In one embodiment, the system receives 3D image data. In other embodiments, the system includes a 3D mapping system, which includes a 3D scanner 191 for image data capture.

FIG. 19 illustrates aspects of preparation of the body part, a foot here, for the image data capture. In order to correct the posture of some patients, a designated foot holding apparatus is used for placing and fixing the foot at the neutral position or any other position in which the caregiver deems the foot should be during gait. In exemplary process, the foot is maintained in that position during image data capture, more specifically a constant height y and distance x from the 3D scanner 191. One embodiment of a system provides a body positioning device 193 to facilitate constant body part position. An exemplary body positioning device 193 provides a rest or containing surface and provides a full field of view of the body part. The depicted body positioning device 193 is a platform, where the foot 192 is placed on its upper surface.

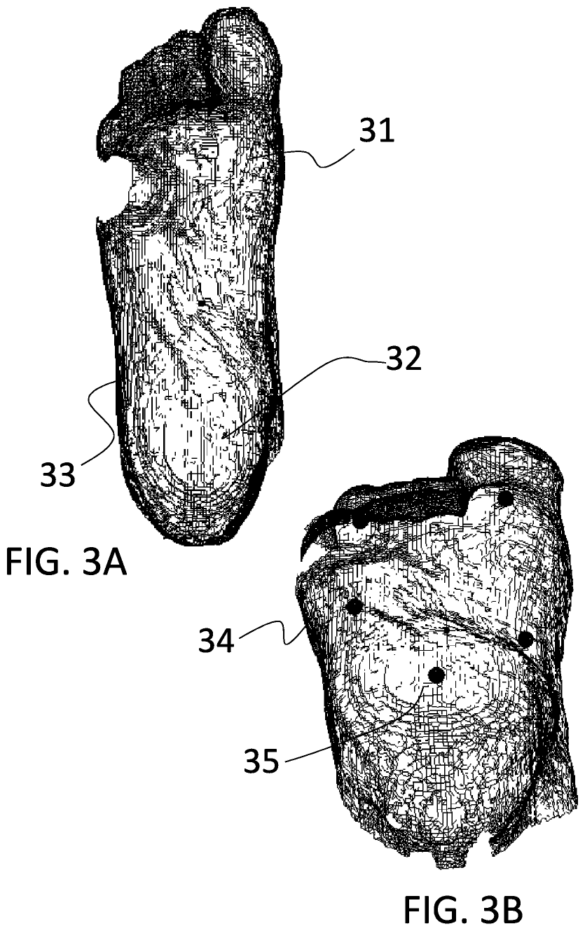

In some embodiments, the process and system uses solely the existing geometry and color information of the body image data to identify the different regions of the foot, such as the toe, metatarsal, arch, and heel regions. FIG. 3a shows a foot with identified anatomical landmarks of the metatarsal joint 31 and the bottom part of the heel 32 on a scanned model of a foot 33. In other configurations of the system and processes, anatomical landmark detection is facilitated by use of markers 35 placed on the foot. Such a configuration is shown in FIG. 3b. A series of anatomical markers 35 is deployed by which are distinguishable by the image data processing subsystem. Exemplary anatomical markers include labels with a glue on one side and color on the other, a marker pen with colored ink, or paint. The color of the pigment should contrast the skin of the body part. Anatomical markers 35 are attached to one or more of the following: left/right/upper/lower toes, left/right/upper/lower metatarsal region, left/right/upper/lower arch, left/right/upper/lower heel region, or other landmarks. FIG. 3b shows the heel mark using an anatomical marker 35 which was placed on the plantar surface of a patients foot 34 to describe the bottom most part of the heel portion.

The image data is captured with a 3D scanner 191. A suitable 3D scanner is one or more depth sensing and red green blue (RGB) cameras. For example, the camera may be a Microsoft Kinect, Primesense, David Laser or other depth sensing cameras. Common depth sensor cameras includes technologies such as laser or IR emitter/receiver pairs. The image data is captured, with the 3D scanner rotating about the foot in exemplary configuration. Representative image data output formats include Standard Tessellation Language (STL), the open geometry definition format (OBJ), or the polygon file format (PLY).

After the system receives the image data file 200, the system maps the landmark anatomical features as well as other features of the foot are mapped 300. In regards to the anatomical features, the anatomical markers 35 are associated with points, typically vertices, of the image data. A representative subsystem for this step is a Blender 3D engine based tool.

As mentioned, the system also maps other features of the foot 300. Clinical conditions of the foot such as diabetic foot, foot ulceration, wounds, infections, osteonecrosis or others may exist in the subject foot. In order to treat the pathologies, the system needs to identify the problematic zones and areas on a patient's foot. All of the information is gathered in the 3D map of dots which represents the patient's foot. As previously mentioned, the 3D scanner 191 may use one or more of the following technologies including projected structured light, projected IR or NIR speckle, laser scanning, triangulation based image analysis, or other 3D mapping technology. To analyze the center points and areas requiring load discharging, the data is analyzed using the geometrical map, the spectral images, and the IR and NIR filtered data for determining “hot spots” or combinations of the above. Additionally, color filtration and differentiation may be employed to aid in detection of such conditions. The zones having identified clinical conditions are marked in the image data. A representative subsystem for this step is a Blender 3D engine based tool.

An orthotic base model is generated, where the orthotic base represents a surface for mating to the corresponding mapped plantar surface, conforming to the generated corresponding mapped plantar surface.

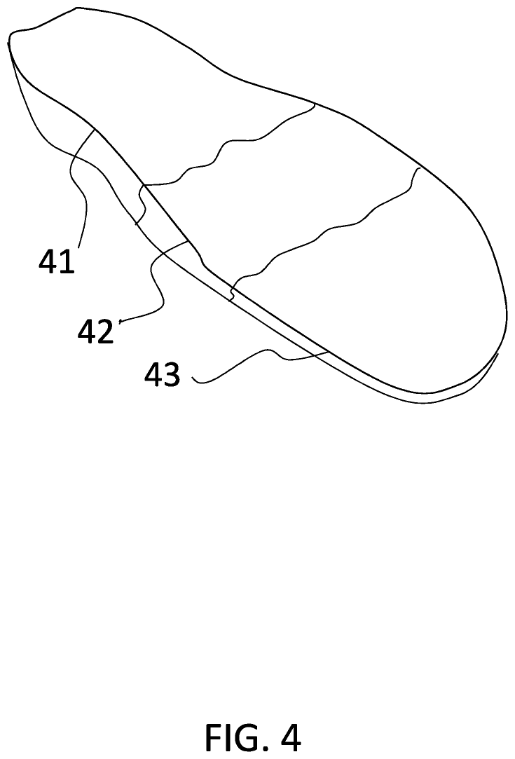

At step 400, the orthotic base model is amended and optimized. The system runs a series of automated amendments as well as receiving manual amendments in order for optimum fit to the required posture and position. In one configuration, the orthotic base model is divided into multiple sections as seen in FIG. 4, where one section 43 of a first angular orientation is continuously joined to a transition section 42 of a second orientation, which in turn is joined to a third section 43 of a third orientation, while the combined sections 41 42 43 remain rigidly joined. The rate of gradient transition of the middle section 42 is proportional to the orientation of the first section 41 and third section 43.

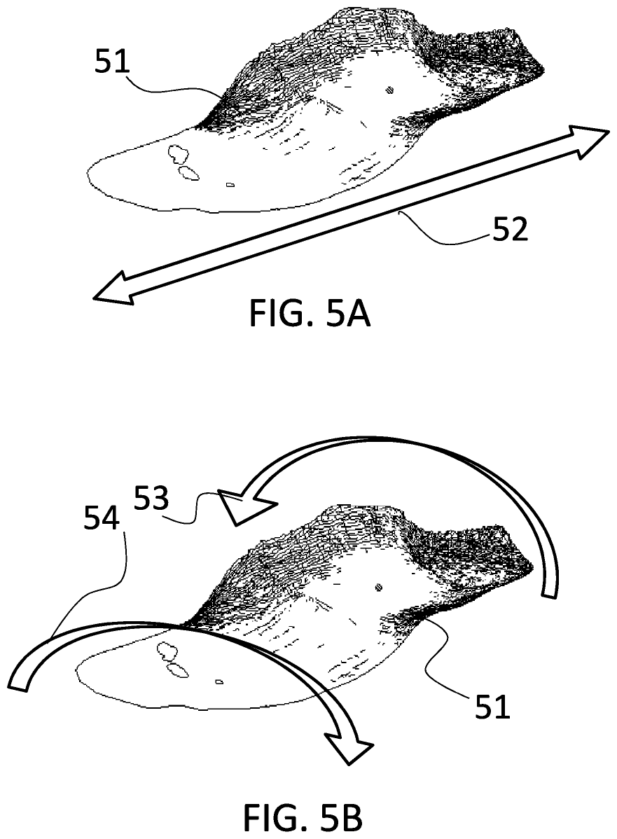

The system automatic performs some amendments and facilitates user manipulation of the orthotic base model. Representative operations include inflation/deflation, smoothing, gradual pulling/pushing operations, stretching, compressing, rotating the representative orthotic base model or sections 41 42 43 thereof. FIGS. 5a and 5b illustrate some of the amendments or transformation which may be applied to the orthotic base model. FIG. 5a shows a longitude stretch 52 operation which is performed on part of a scanned surface 51. FIG. 5b shows a twisting manipulation which is typically used for fixing posture related angular deformations in patients. The twisting can be performed on the heel end 53, the toe end 54 or combination of the two. The deformations as well as stretching may be performed manually by the technician, clinical or operator or automatically by the system in order to enable the subtalar neutral position. In addition to these operators, the system allows flattening of specific portions of the foot, to allow a comfortable fit of the system in a standard shoe.

In image 6A, the scanned object 61 has been flattened in the Z axis direction 63 to allow a flat surface at the distal portion of the orthotic. This action can also be performed according to the gradient principal as shown in FIG. 4.

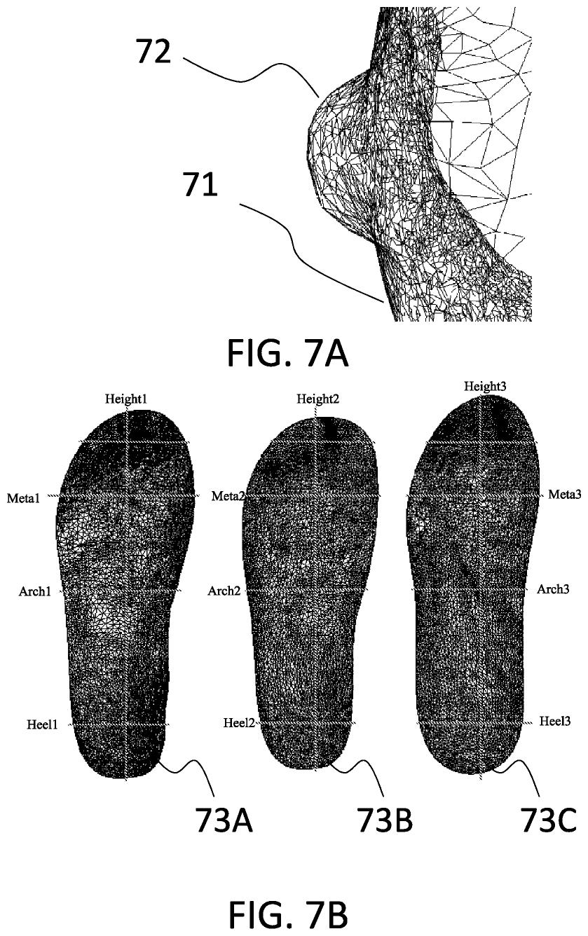

Some additional features the system includes, allow digitalization of typically manual sculpting like actions. FIG. 7a shows an example of such activity, where a specific area on the surface 72 of the scanned body 71 is inflated. Additional operators include pulling/pushing, smoothing, flattening and additional sculpting operators. By allowing the operator these digital sculpting tools, he can manipulate the scanned object just as he would do to the cast or insoles using real physical sculpting tools.

Once the 3D geometry of the desired orthotic is determined, the insole contour is used to cut the orthotic in the correct shape for a given shoe. One configuration of the includes a repository of insoles for shoes varied by toe region relative width and height, metatarsal region relative width and height, arch region relative width and height, heel region width and height. FIG. 7b shows a subset of of many contour shapes which are contain therein and fit different kinds of feet, length and widths. The system can use existing contours such as the ones shown in image 73a, 73b and 73c and amend via scaling these to the correct show size for a particular patient. In addition, the system scanner can also scan the specific contour of a patient's existing insole and create new contours fitting specifically to the application.

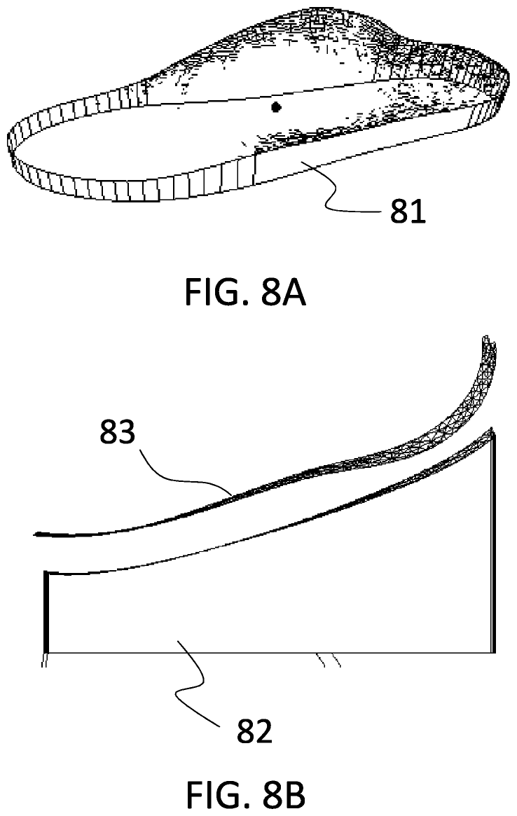

Once the surface has been defined as well as the contour, the orthotic will receive volume and thickness values as shown FIG. 8a. The thickness as well as hardness level of the material can be entered manually or calculated automatically according to the patient's detail and condition as they were input into the system.

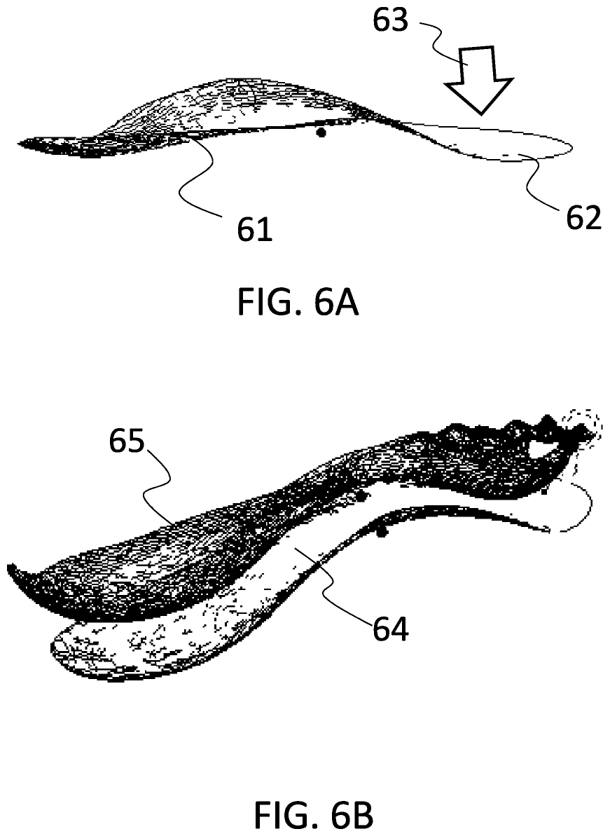

The system contains additional overlay interfaces which for comparison which limit errors and further optimize the fit of the orthotics. In exemplary display, the overlays are presented in an opaque or semitransparent manner. FIG. 6b shows an example of such tool, where a copy of the original foot scan 65 is projected on top of the amended surface of the orthotic 64. This tool allows comparison and evaluation of the amendments made on the original surface and the way they affect the fit to the patient's original scan. Additional overlay interfaces included in the system include a cross section view. As seen in FIG. 8b the section view cuts along one of the main axes through the designed orthotic 82 as well as through the original scan copy 83. This allows local evaluation of the design as well the fit of the design to the plantar surface.

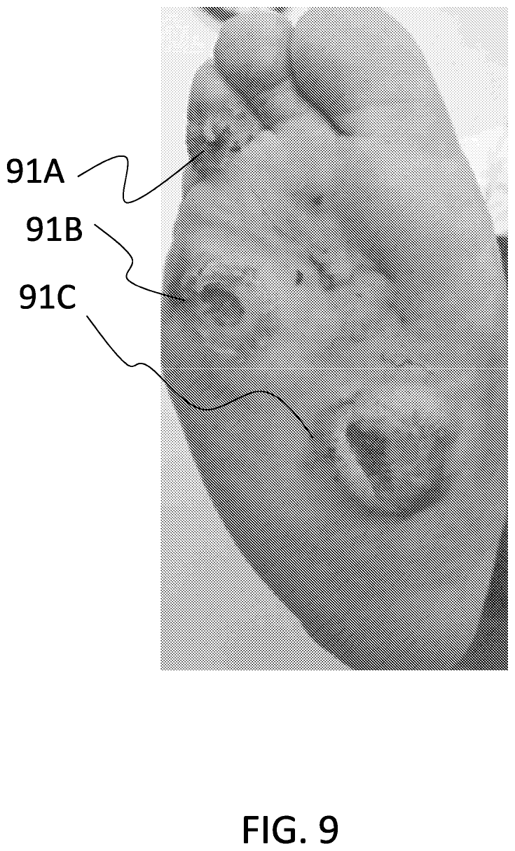

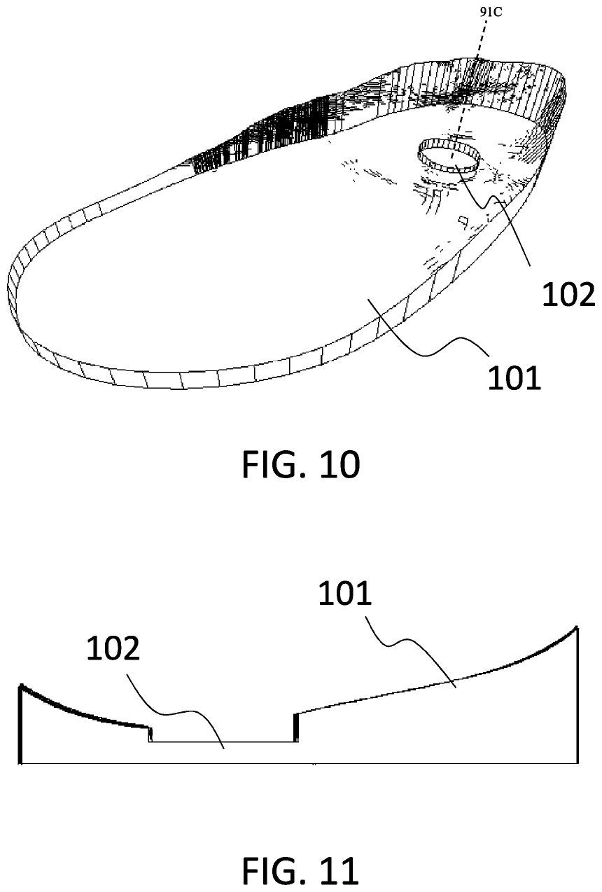

At step 400, the orthotic base model is optimized. Optimizations include load offset from affected sites and drug delivery to affected sites. FIG. 9 depicts an example of a diabetic foot of a patient which includes ulcers and wounds at locations 91A, 91B and 91C. By analyzing this data employing the steps previously disclosed, the location of the ulcerations and wounds were mapped. In this configuration, corresponding holes 102 are placed in the body of the orthotic as shown in an example in FIG. 10. A hole 102 can protrude fully or partially into the body of the insole 101 to allow the wounds and ulcer segments to remain load free and promote healing. The hole 102 can also be seen in cross section in FIG. 11.

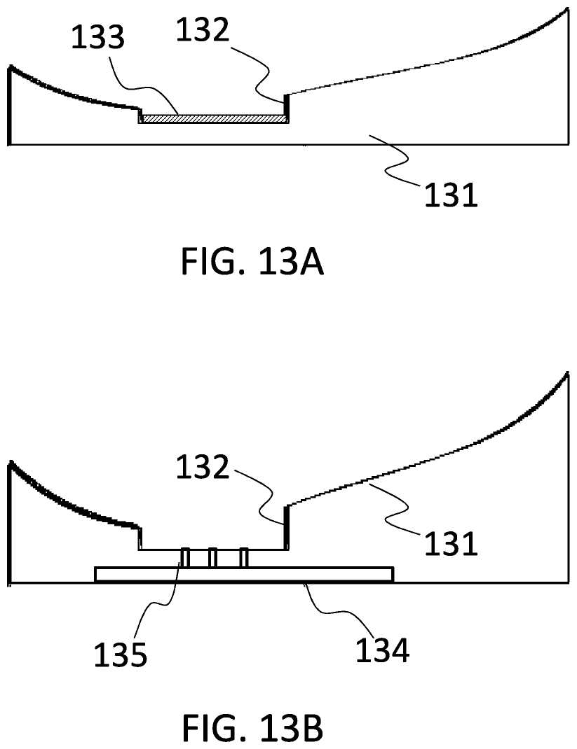

In some embodiments of the present invention, the surface of the orthotic may be fully or partially coated with anti-bacterial, anti-fungal or controlled drug release coating. An example for this embodiment can be seen in FIG. 13a. A recess 132 is disposed in the body of the orthotic 131 corresponding to a wound site 91a 91b 91c. Substrate 133 represents a layer of drug coating, anti-bacterial coating, or other coating which can be locally positioned to treat the wounds and improve the healing process. An additional configuration of the current embodiment, shown in FIG. 13b, includes a plurality of exit channels 135 in fluid communication with a remote reservoir 134, presenting a fluid exit from the wound site to the reservoir, allowing the healing surface to remain dry and thus promoting healing.

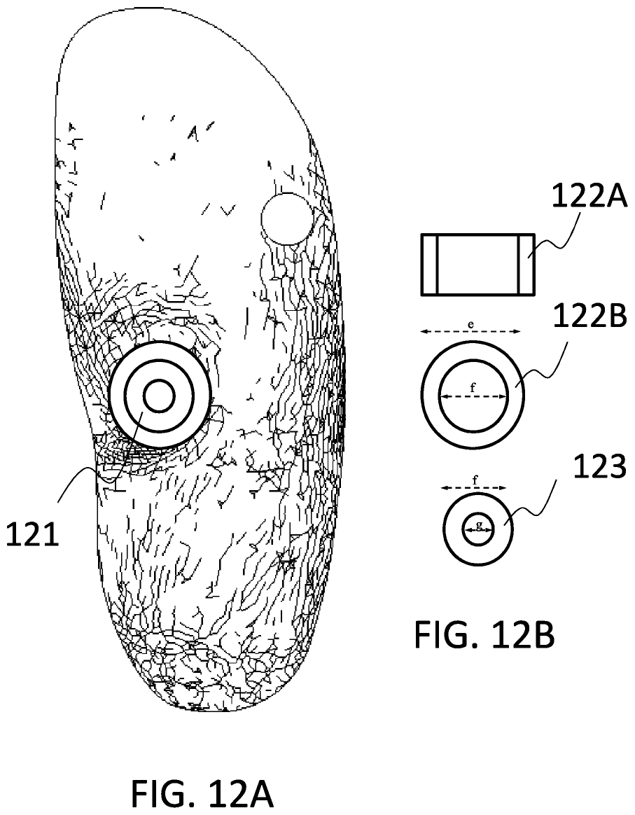

As healing of the wound sites commences, the width of the wound site 91a 91b 91c decreases. FIG. 12a illustrates an example orthotic built with the system which includes two recesses 102 dimensioned to accommodate ulcers and wounds. A plurality of concentrically disposed, successively smaller plugs 121 are inserted in the recess 102. Each plug 121 has an outer width sized to the fit in the adjacent plug (or the recess 102 in the case of the outer plug 121). Each plug 121 has an inner opening sized for receipt of a successively smaller plug 121. The shown plugs 121 are toroidal having successive outer widths of e and f and successive inner widths off and g, which can be seen in separate in FIG. 12b, plugs 122b and 123. In use, the plugs 122b 123 are inserted into each other and into the depression zones and allow gradual shrinking of the depression diameter and support for the wound as it heals and shrinks. These elements can be formed from the same materials as the orthotic or from different material of various hardness levels such as silicone or ethylene vinyl acetate (EVA).

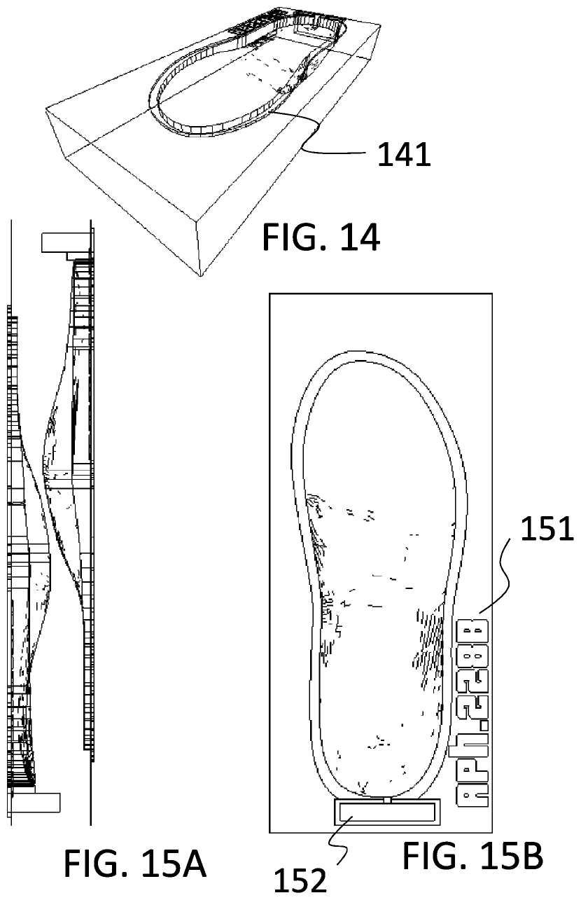

Once the system has finalized the design of the specific orthotic, the negative impression of the orthotic is transformed into a model of a mold. An example for such mold can be seen in FIG. 14. The mold may contain a lip 141 formed around the perimeter of the cavity which protrudes to a height 0.1 to 5 mm over the surface of the mold. This lip will contact the flat surface of the mold during injection molding and ensure a tight closure is achieved for injection. The molds may be milled on two sides of a single block as shown in FIG. 15a to save material and volume. In this case, a single patient's two orthotics can be milled and saved on a single block. Additional details which may be included in the molded blocks include an “air box” cavity 152 located behind the heel of the orthotic as shown in FIG. 15a. This “air box” is aligned to the material inlet in the injection process and therefor prevents from the deformations and discolorations associated with the material inlet from being present on the surface of the orthotic while injected. Detail 151 in FIG. 15B describes the details of a patient which may also be milled on the surface of the mold. These details can contain some of the following, but not limited to these: the patient name, initials, right or left foot, the volume of the cavity for injection, the required hardness level, the weight or any additional information relevant to the orthotic or the injection process.

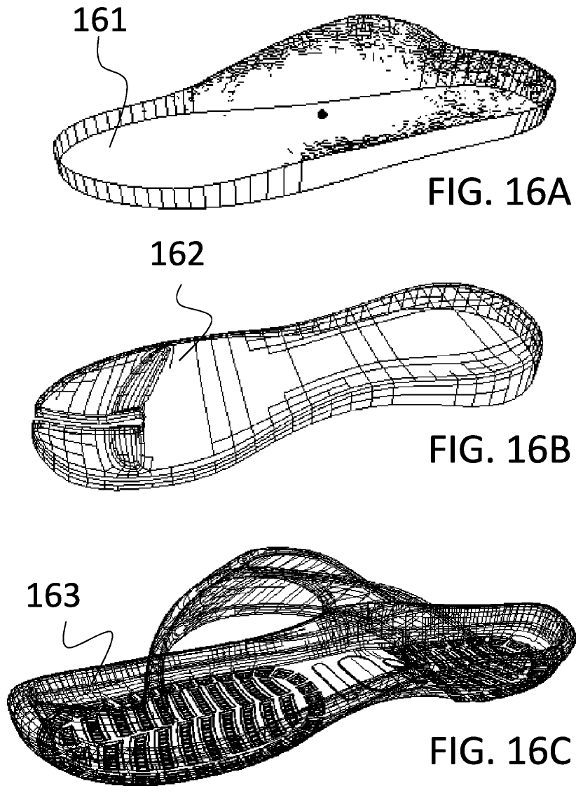

Open toe footwear present additional manufacturing and use problem compared to closed toe footwear. FIGS. 16a, 16b and 16c depict an example of an embodiment of the current invention which describes the design and manufacture of a personalized custom flip flop. In the current embodiment the orthotic is designed according to the flow chart described in FIG. 1. The curve used to cut the surface to the correct contour is designed according to a shape as depicted in FIG. 16b but not limited to this shape. This shape fits exactly into a cavity of the same shape which is located in the body of the flip-flop in detail 163 of FIG. 16c. the cut to shape orthotic is inserted and adhered or chemically or heat bonded into the cavity forming a flip-flop with a personalized orthotic surface. The flip-flop may include a strap which can be connected or disconnected from the heel portion of the flip flop and creates better support and gait to the users.

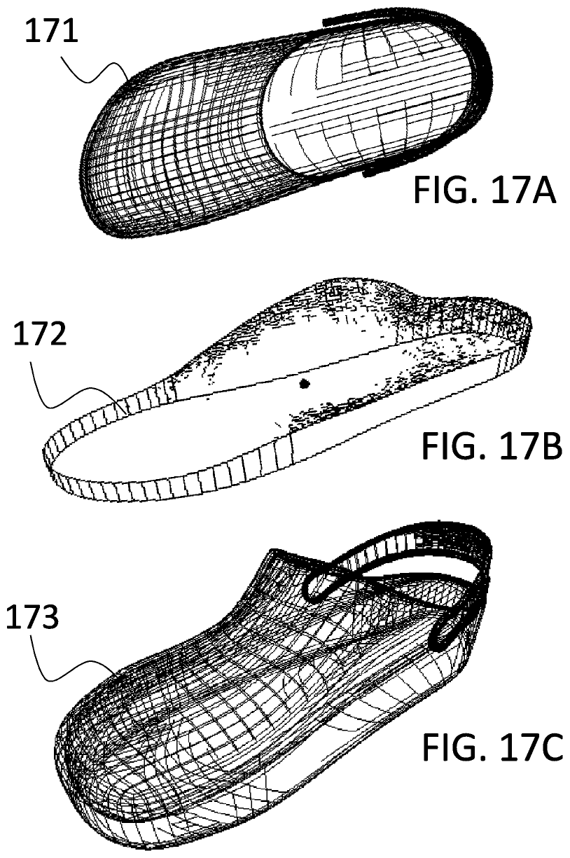

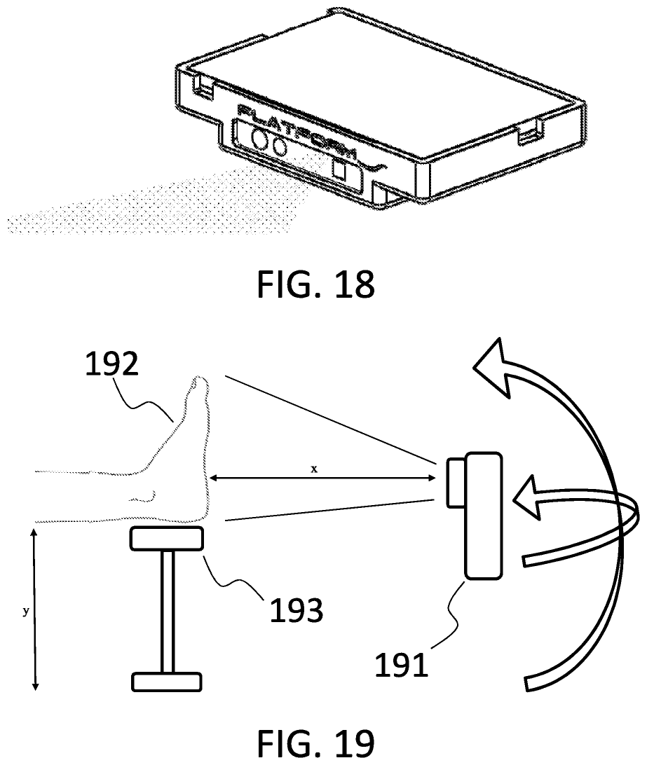

FIGS. 17a, 17b, and 17c relate to another part of the current invention, related specifically to personalized clogs and slippers of various configurations, such as Crocs. FIG. 17a shows an example clog design which includes a cavity formed specifically to include the orthotic designed according to the flow chart described in FIG. 1. The orthotic, which example can be seen in detail 172 of FIG. 17b fits to the contour of the clog 171 and can be adhered into place or chemically or heat bonded. Alternatively, the clog 171 can host more than one orthotic sole 172 and these can be replaced according to different colors, materials, form factors or according to any other consideration that may require alternative orthotics. An additional embodiment of this invention includes the forming a personalized clog using a single molding process as seen in FIG. 17c. The one part clog injection 173 can be formed by replacing a piece of the core part of the injection mold to allow the exact fit to a patient's foot. The embodiments described in FIGS. 16a-16c as well as 17a-c describe a process of the invention for creating different personalized footwear. These are not limited to flip-flops or clogs which are described herein but are also relevant to customized sandals, slippers and soles for closed shoes personalized for each consumer.

It should be appreciated that the disclosed 3D scanning, amending, and manufacturing processes can be applied to other product such as but not limited to personalized intra-ear earphones, off loading braces, orthopedic support braces for the knee, ankle, elbow, back, neck or any other body ligament including braces with mobility, semi mobile or fully stabilizing braces, and the earphone piece.

FIG. 2 represents a typical yet not limiting flow chart of the current invention. The process starts with the scanning of the patients foot using a 3D mapping unit 21. The data is uploaded via the internet to the laboratories or alternatively processed locally taking into consideration the mapping factors as well as personal details from the customer. The negative impression of the design orthotic is then carved on a block or made using additive manufacturing technologies 24 into a mold 25 used for injection molding of one of the final products, including personalized orthotics 26, flip flops 27, clogs 28, or shoe soles, slippers or application non-related to the feet such as fixation aids for head, hand, legs or other part of the body as well as custom apparatus such as chairs, handles and more.

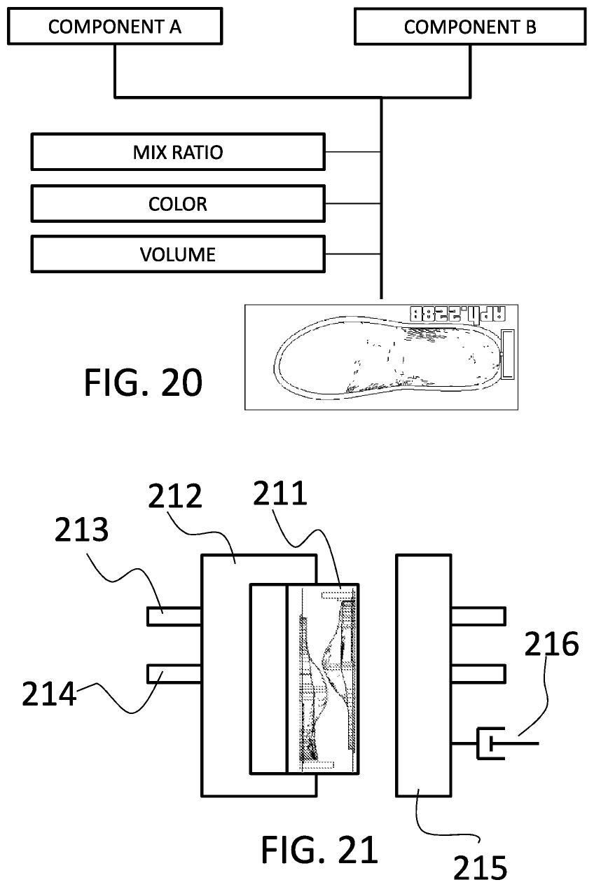

Another part of the invention related to the controlled process of injection molding of the orthotic insoles, soles or personalized shoes. FIG. 20 describes a flow chart of all the controlled parameters which can affect the final product produced in the injection molding process. While in some preferred embodiments two components or more are mixed together during the injection, the user can control the injection volume, the pigment affecting the color of the product as well as the mix ratio which controls the hardness level of the product. These parameters may be picked manually by the technician or operator or be automatically calculated according to the system inputs. The parameters may be milled on the surface of the mold block to facilitate the insertion into the injection system by the end user. FIG. 21 describes an example injection molding tool according to one embodiment of the current invention. In this setup the mold housing includes a base block 212 which includes a cavity according to the dimensions of the blocks. These blocks 211 are interchangeable according to the specific mold for each client. The housing block 212 includes a heating system 213 which could be electric or using warmed fluids as well as a cooling system 214. The second part of the molding system includes a pneumatically or mechanically moving plate 215. This plate includes the material inlet as well as a mechanical or pneumatically controlled closure system 215.

Although the invention has been described in conjunction with specific embodiments thereof, it is evident that many subsystems, subprocesses, alternatives, modifications and variations will be apparent to those skilled in the art. Accordingly, it is intended to embrace all such subsystems, subprocesses, alternatives, modifications and variations that fall within the spirit and broad scope of the appended claims.

### EXAMPLES

Aspects of the present teachings may be further understood in light of the following examples, which should not be construed as limiting the scope of the present teachings in any way.

### Example 1: Producing a Custom Orthotic Using the System Described Herein

Healthy patient, 34 years of age, 190 cm of height, 85 Kg weight. Engages in sports 1-3 times a week including running and basket-ball. Patients complaints and appears to be in discomfort. Diagnosed with plantar fasciitis, Functional hallux limitus.

After physical examination the patient was seated in a chair and the feet were positioned in the subtalar neutral position. The position of the foot was fixated using the designated holder described in FIG. 19. Once the foot was positioned the 3D mapping was performed, resulting with a 3D scan of the plantar surface of the foot in the subtalar joint neutral (STJN) position. This was repeated for the second foot. All of the patient's information including personal, clinical and diagnosis was uploaded through the system's portal together with the patient's scans. Additionally the existing inserts from the sports shoes which are used by the patient normally were mapped and uploaded to the portal as well. The 3d orientation and modifications were performed by the system and the orthotic contour was cut according to the scanned insole in order to best fit into the sports shoes. The parameters inserted about this patient suggested a medium hardness level for this patient, referred to by the system as “B”. After the orthotic 3D model was created and the volume was calculated, the mold model was fabricated including these parameters written on the mold, as seen is FIG. 15B detail 151. This file was sent to the automated CNC milling machine which fabricated a two sided mold block specific to the patient's geometry. The block was inserted into the mold housing similar to the one described in FIG. 21 where each side was injected with two component foamed polyurethane material. The mass of each component as well as ratio were determined according to the parameters calculated and written on the body of each side of the mold, in this example 80 grams of component A and 50 of component B. After the injection molding process was completed, the orthotics were tested to the correct hardness level, in this case 15 Shore A which is within the “B” hardness level limits which are 14 to 16 Shore A.

### Example 2: Producing a Custom Orthotic for Diabetic Foot Using the System Described Herein

Diabetic Patient, 37 years of age, 185 cm of height, 105 Kg of weight. Suffers from severe diabetic related neuropathy in both feet, has multiple wounds and ulcers on each of his feet. Suffers from Charcot foot. Has a history of wound infection which have put his feet in danger of amputation. Uses diabetic's footwear.

After diagnosis of the patient, his feet were placed in the foot holding unit and scanned at the STJN position. The scan included spectral information as well as 3 axis geometrical information for each vertex on the surface of the patient's foot. The scans and patient information were sent via the internet to the systems portal for diagnostics. While analyzing the scanned surface, three ulcer location were diagnosed on the plantar surface of the patient's foot, as seen in FIG. 9 details 91A, 91B and 91C. The insole design was built to support the foot around the wounds while allowing zero weight bearing at the wounded and ulcer regions of the foot. The patient received a treatment in which he had to return for follow up on the wound conditions once a month. Each time he came, as the treatment improved the condition of his wounds and reduced the size of these, he had received a new pair of orthotics with the same geometry only with smaller diameter strain release portions, as seen in an example on FIGS. 12a and 12b. The hardness level of this patient's insoles was determined as according to the system and therefor injection molding parameters were set to 11 Shore A, inside the A range which is 10-12 Shore A.

### Example 3: Producing Custom Clogs Using the System Described Herein

Healthy patient, male 55 years of age, 175 cm of height, 75 Kg weight. Works as surgeon at a hospital, requiring up to 12 hour of standing every day. Patients uses orthotic insoles while engaging in sports but wares clogs while in the operating room. Generally healthy, has a pes cavus foot condition, history of plantar fasciitis.

The patient was scanned using the system at the STJN position using the equipment and setting as described in example 1 above. The scans were aligned and manipulated using the system software after being uploaded to the portal. The contour used for trimming the boundary line was a specific contour line which fits the cavity of the custom clog's design, such as the once described in detail 172 of image 17B. After being manufactured using the process described in the above examples the orthotic was adhered to a pair of size 11 orthotic clogs, forming a water tight, one piece like clog. The orthotic clog was injected with a two component polyurethane foam which included silver particles in order to reduce the chance for infection while using the clogs. In addition, the same orthotic was also trimmed a second time using the software, this time according to the contour which best fits the flip flop orthotics, such as the one described in detail 162 of FIG. 16B. This orthotic was manufactured the same way and integrated into a custom flip-flop pair which was also supplied to the patient.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A system for designing orthopedic devices, custom orthotics or personalized footwear based on computerized design software adapted to adjust the scanned information into a 3D model of the device substantially ready to production, the system comprising an imaging module which uses image recognition to identify different anatomic parts of the foot to allow the design of the orthotics, and a human interface that allows showing the original scan of the foot at an opaque or semitransparent manner to allow visualization of the way the foot is going to fit and be supported by the designed custom orthotic.

2. The system of claim 1, further adapted to designing 3D models or objects which are specifically adjusted to fit a certain portion of the human body.

3. The system of claim 1, further comprising a marker system used for marking segments on the skin of a patient which are recognized using the system and are used for the identification of the specific organ parts as well as required posture, the marker system comprising stickers which are easily recognized using the 3D scanner according to color information, specified geometry, and/or light absorption properties.

4. A manufacturing technique for building custom orthotic insoles or personalized sandals or footwear soles using injection of a foamed or soft material into a mold, the technique comprising heating circles and/or cooling tubes; and receiving inserts to a fixed flat side and cavity built to receive inserts according to the geometry of a specific orthotic.

5. The manufacturing technique of claim 4, wherein the personalized sandals or footwear soles comprise custom orthotic insoles designed to treat foot wounds and ulcerations by correct distribution of the weight of a patient along the plantar surface of the foot and by including strain release depressed areas/holes in the orthotic, the insoles comprising: a plurality of materials of different hardness levels, wherein the soft materials are located under the wounds allowing load reduction from these points.

6. The manufacturing technique of claim 4, wherein the personalized sandals or footwear soles comprise a custom orthotic flip-flop built according to specific mapping of a person's foot geometry in order to fit the plantar surface and supply arch support, the flip flop comprising a patient's foot scan was performed at a subtalar joint neutral position to correct the posture on a patient's foot.

7. The manufacturing technique of claim 4, wherein the personalized sandals or footwear soles comprise custom orthotic clogs are built according to specific mapping of a person's foot geometry in order to fit the plantar surface and supply arch support, the clogs comprising a patient's foot scan was performed at a subtalar joint neutral position to correct the posture on a patient's foot.

8. The manufacturing technique of claim 4, wherein the personalized sandals or footwear soles comprise custom orthotic sandals are built according to specific mapping of a person's foot geometry in order to fit the plantar surface and supply arch support, the sandals comprising a patient's foot scan that was performed at a subtalar joint neutral position to correct the posture on a patient's foot.

9. The manufacturing technique of claim 4, wherein the personalized sandals or footwear soles comprise custom footwear designed and manufacturing specifically according to a customer's body geometry, the shoes comprising a sole made from an insert mold specific to a patient's geometry, and wherein the sole is combined with the sole molds to allow multiple colors, material properties and geometrical patterns.

10. A process for creating customized orthotics, the process comprising:
receiving a 3D file of a foot, said 3D file comprising the metatarsal region, the arch region, and the heel region;
detecting and assigning position data in said 3D file for said metatarsal region, said arch region, and said heel region;
generating a base orthotic model, where said orthotic base model represents a surface for mating to the corresponding mapped plantar surface, said base orthotic model conforming to the mapped plantar surface.

11. The process of claim 10 further comprising providing a body position device, operable to facilitate a constant foot position during 3D image data capture.

12. The process of claim 10 further comprising providing a marker pen and marking the metatarsal region, the arch region, and the heel region.

13. The process of claim 10 further comprising providing paint and marking the metatarsal region, the arch region, and the heel region.

14. The process of claim 10 further comprising providing label and marking the metatarsal region, the arch region, and the heel region.

15. The process of claim 10 further comprising providing a 3D scanner comprising a depth and color camera.

16. (canceled)

17. The process of claim 10 further comprising detecting and assigning position data in said 3D file for wounds on said foot.

18. The process of claim 17 further comprising detecting using one or more of hotspot detection and color differentiation.

19. The process of claim 17 further comprising:
modifying said base orthotic position to provide recesses corresponding to the position of said wounds;
applying a healing agent as a substrate in said recesses; and
providing an exit channel in fluid communication with said recess and a remote reservoir.

20. The process of claim 10 further comprising:
presenting an interface for manipulation or verification of said base orthotic model, wherein said interface is adapted to enable one or more functionalities including inflation or deflation operation, a smoothing operation, a stretch or compress operation, and a rotation operation.

21. The process of claim 10 further comprising generating the negative impression of the orthotic model and transforming into a model for a mold.
